(19) United States
(12) Patent Application Publication
(10) Pub. No.: US 2010/0030060 A1
(43) Pub. Date: Feb. 4, 2010

(54) Scope, Vision

(76) Inventor: Wanda Sublett, Grandbury, TX (US)

Correspondence Address: Wanda Sublett, 3501 MCCreary Road, Grandbury, TX, 76049, US

(21) Appl. No.: 11/550,404

(22) Filed: Aug. 4, 2008

(51) Int. Cl. A61B 5/055

(52) U.S. Cl. 600/410

(57) ABSTRACT

The VISION SCOPE allows claustrophobic individuals to enter an open or closed Magnetic Resonance Imaging (MRI) machine without claustrophobic tendencies and view the outside areas while undergoing testing without interfering with the magnetic field created by the MRI machine.

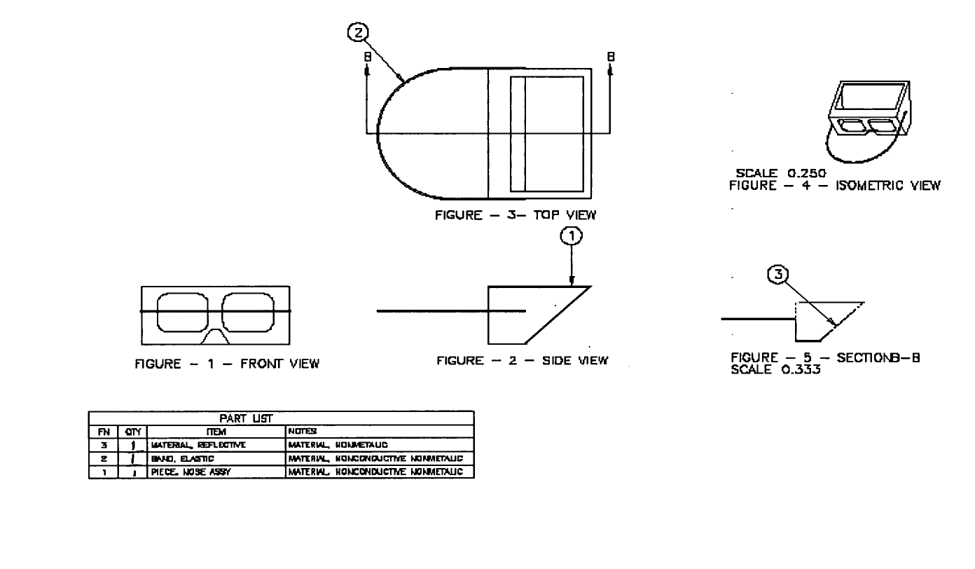

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Our field of endeavor to which the VISION SCOPE pertains is the medical and laboratory field of endeavor. The VISION SCOPE allows claustrophobic individuals to enter a closed or open Magnetic Resonance Imaging (MRI) machine alleviating claustrophobic tendencies and allowing the individual to view the outside areas while undergoing this procedure.

### BRIEF SUMMARY OF THE INVENTION

Many people entering a closed or open MRI machine display claustrophobic tendencies which often require sedation, removal from the machine, rescheduling and an expenditure of medical personnel and equipment resources. Our research indicates that a significant number of individuals requiring a closed MRI must be sedated prior to the MRI procedure as well as being removed from the machine more than once prior to completion of this procedure.

The use of the VISION SCOPE was demonstrated to alleviate the claustrophobic tendency and allows the MRI to be completed without removal from the machine. The non metallic nature of the paper and mirror sheeting does not affect functionality of the MRI machine.

1. I claim as my invention a Scope, Vision which allows claustrophobic individuals to enter an open or closed Magnetic Resonance Imaging (MRI) machine without claustrophobic tendencies and view the outside areas while undergoing testing.

2. Our research indicates that a minimum of 30% of individuals requiring a closed MRI must be removed from the machine prior to completion of the procedure.

3. The non metallic nature of the mirror sheeting does not affect functionality of the MRI machine.

4. The use of the Scope, Vision alleviates the claustrophobic tendencies for individuals, by allowing them to view the outside areas thus enabling total completion of the MRI.

5. Our research indicates some individuals requiring a closed MRI required medication prior to the MRI procedure. In some cases the use of the Scope, Vision has eliminated the use of medication.

6. The availability lessens the pre-MRI stress of claustrophobic individuals.

7. Use of the Scope, Vision will allow laboratories to be more efficient and effective by scheduling more patients and having less down time in the testing process.

8. Cost of the Scope, Vision is minimal as compared to the machine usage time and cost per individual.
